(19) United States
(12) Plant Patent Application Publication
(10) Pub. No.: US 2002/0157146 P1
(43) Pub. Date: Oct. 24, 2002

(54) GAURA LINDHEIMERI PLANT NAMED 'CRIMSON BUTTERFLIES'

(76) Inventor: Howard Bentley, Victoria (AU)

Correspondence Address: CHRISTIE, PARKER & HALE, LLP, 350 WEST COLORADO BOULEVARD, SUITE 500, PASADENA, CA, 91105, US

(21) Appl. No.: 09/837,406

(22) Filed: Apr. 18, 2001

(51) Int. Cl. A01H 5/00

(52) U.S. Cl. PLT/226

(57) ABSTRACT

Described as a new Guara Lindheimeri plant having short narrow, crimson colored leaves growing on a very compact plant with short racemes.

### BACKGROUND OF THE INVENTION

The present invention relates to a new Guara Lindheimeri plant having the varietal denomination ‘CRIMSON BUTTERFLIES’. ‘CRIMSON BUTTERFLIES’ was discovered as a seedling variant of the variety Guara Lindheimeri ‘SISKIYOU PINK’ in a controlled planting.

### SUMMARY OF THE INVENTION

The new variety of a Guara Lindheimeri plant is an evergreen perennial shrub which has a compact, dense, spreading, growing habit distinguished by its leaves, flower color and size. Asexual reproduction of the new variety by vegetative cuttings in Wonga Park, Victoria, Australia has demonstrated that the distinguishing characteristics are transferred through succeeding generations.

### COMPARISON WITH PARENT

The new variety differs from its parent, ‘SISKIYOU PINK’, in being more compact and dense and having a shorter raceme length. The new variety also has a shorter and more narrow leaf length than its parent variety and has a different leaf color.

### BRIEF DESCRIPTION OF ILLUSTRATIONS

The accompanying illustrations depict specimen plants of the new variety in color as true as possible in color photographic illustrations of this nature.

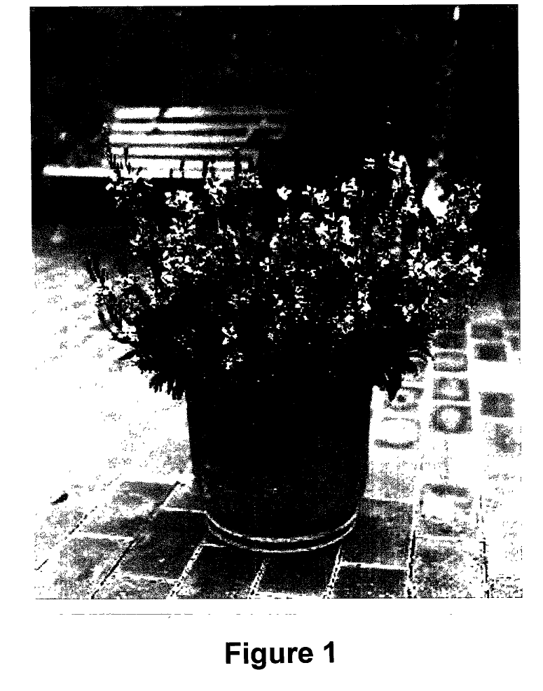

FIG. 1 shows the plant of the new variety;

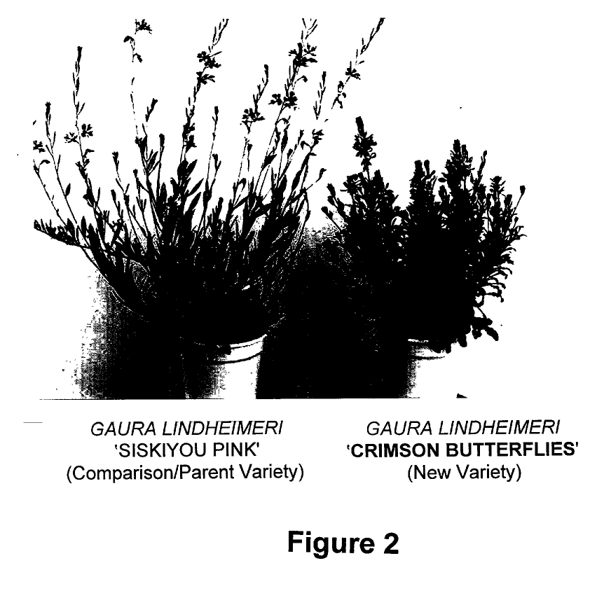

FIG. 2 shows the new variety aside its parent variety; and

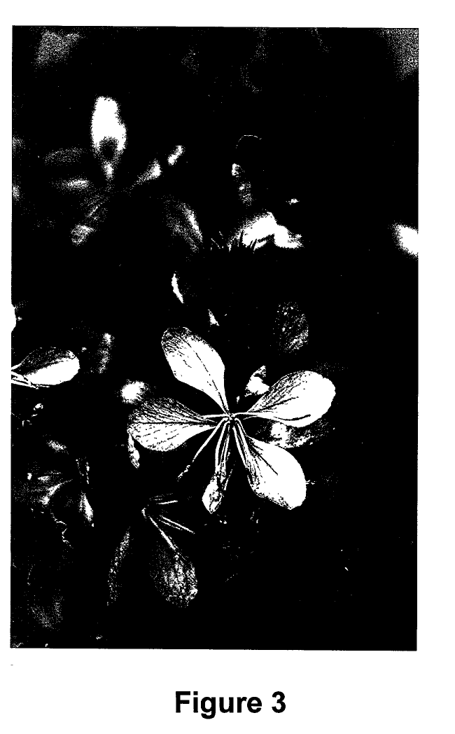

FIG. 3 shows a close up of the flower of the new variety.

### DETAILED DESCRIPTION OF THE NEW VARIETY

The following is a description of the new variety taken from plants grown out-of-doors in Wonga Park, Victoria, Australia during the month of April. Color designations used herein are from the Royal Horticultural Society Color Chart (“RHSC”).

Plant: The plant grows in a spreading habit with compact, dense growth.

Foliage:

Leaflet.—Size: About 52 mm. long and about 11 mm. wide.

Quantity: Dense.

Color.—Leaflet base: Near 187A Leaflet tips: Near 183C.

Shape.—Leaves are linear to lanceolate ovate to narrow ovate.

Texture.—Upper side: Smooth with fine pubsescents Under side: Smooth with fine pubsescents.

Veination.—Reticulate.

Edge.—Slight undulate margins observed.

Wood:

Bark.—Bark is smooth with fine pubsescents.

Flower:

Arrangement.—Raceme length very short; near 183A; tubular, diameter about 30 to 31 mm; 4 to 5 petals, near 53D;

Winter hardiness: Winter hardy to between 7° and 5° C.

Reistance to disease: Good, observed to be generally disease resistant.

Breeding: First observed as open-pollinated seedling in trial bed among a crop of seedlings raised from the parent variety. This compact variant was isolated in 1997 and has since been selected through several cycles of selection to development.

Reproductive organs:

Arrangement.—Stamens, filaments and anthers are regularly arranged at the base, but bunched in a pendula formation.

Filaments.—White to pink dependant on age.

Anthers.—Crimson.

Pollen.—Yellow.

Styles.—Thin, singular, medium length.

Stigma color.—Between pale green and cream colored.

Hips.—Shape: Oval to linear Size: Small with no seed protrusion

I claim:

1. A new Gaura Lindheimeri plant of the variety substantially as shown and described.
